(19) United States
(12) Patent Application Publication
(10) Pub. No.: US 2002/0194638 A1
(43) Pub. Date: Dec. 19, 2002

(54) Vector for genetically modifying non-human animals

(76) Inventors: Kangsheng Wang, Rowland Heights, CA (US); Jin Qian, Walnut, CA (US); Chao-Kuen Lai, Houbi Shiang (TW)

Correspondence Address: LYON & LYON LLP, 633 WEST FIFTH STREET, SUITE 4700, LOS ANGELES, CA, 90071, US

(21) Appl. No.: 10/039,231

(22) Filed: Dec. 31, 2001

Related U.S. Application Data
(63) Continuation-in-part of application No. 09/537,861, filed on Mar. 28, 2000.
(63) Continuation-in-part of application No. 09/781,046, filed on Feb. 8, 2001.

(51) Int. Cl. A01K 67/00

(52) U.S. Cl. 800/21; 800/5; 800/6

(57) ABSTRACT

A non-liposome based linker such as an antibody is disclosed for use attached nucleic acid molecules to sperm cells. The antibody is characterized by having binding affinity to a sperm cell and wherein the sperm cell bound with the antibody retains the ability to fertilize an oocyte. A resulting sperm-linker-nucleic acid complex may- be used to generate genetically modified animals and cells by in vitro or in vivo fertilization with an egg cell. Introduction of exogenous DNA using this method in various animals (mice, pigs, chickens, and cows) are disclosed. Functional screening of gene with unknown function may also be performed using the disclosed method.

### PRIORITY

This application is a continuation-in-part of co-pending U.S. application Ser. No. 09/537,861, filed Mar. 28, 2000, and co-pending U.S. application Ser. No. 09/781,046, filed Feb. 8, 2001. Both applications are hereby incorporated by reference in its entirety as if fully set forth herein.

### FIELD OF INVENTION

The present invention relates to the field of genetic modification and involves the introduction of exogenous DNA into non-human animals.

### BACKGROUND OF THE INVENTION

Efficient genetic modification of animals, especially in higher mammals, has been a major goal of researchers in the biotechnology field for the last two decades. Not only can genetic modification of animals advance our understanding of genes and gene-functions in multi-cell organisms, it can also serve useful applications in the bio-agricultural industry. Examples of these applications include raising livestock with desired characteristics such as faster growth rate, production of therapeutic proteins in milk, or even the generation of more “humanized” organs from animals for use in animal to human xenotransplantation.

Current techniques to modify the genome include microinjection of foreign DNA into the pronuclei of fertilized eggs, delivery of foreign DNA into embryonic stem cells in vitro or blastomere cells in vivo through lipid-based agents, electroporation, or viral infection. Aside from mice, however, current techniques have been reported to have had limited success in higher or larger animals. The microinjection technique, for example, has been reported to be technically very demanding and requires the use of highly sensitive and expensive equipment.

The viability of embryos after microinjection has also been reported to be very poor. Wall, R. J., et. al. (1992) Making Transgenic Livestock, Genetic Engineering on a Large Scale, Journal of Cellular Biochemistry, Vol. 49, pp. 113-120. This has led researchers in the field to investigate alternative and easier ways of delivering genes into an animal.

In 1989, Lavitrano, M., et. al. reported that simply incubating foreign DNA with mice's sperm cells and effecting fertilization in vitro could lead to genetically modified mice. Lavitrano, M., et. al. (1989) Sperm Cells as Vectors for Introducing Foreign DNA into Eggs—Genetic Transformation of Mice, Cell, Vol. 57, pp. 717-723. Characterized as the “cold fusion” equivalent in biotechnology, this report generated much excitement in the field. Birnstiel, M., et. al. (1989) Dangerous Liaisons: Spermatozoa as Natural Vectors for Foreign DNA?, Cell, Vol. 57, pp. 701-702. Those skilled in the art, however, are reported to remain skeptical even to this day about the Lavitrano's report since a number of researchers in the field have reportedly failed to repeat the experiment. Brinster, R., et. al. (1989) No Simple Solution for Making Transgenic Mice, Cell, Vol. 59, pp. 239-241; Smith, K. (1999) Sperm Cell Mediated Transgenesis: A Review, Animal Biotechnology, Vol. 10(1&2), pp. 1-13.

Over the last decade, efforts have continued to explore the use of sperm cells as a vector for mediating gene transfer in animals. Researchers have elucidated that sperm cells have the inherent ability to internalize foreign DNA. Francolini, M., et. al (1993) Evidence for Nuclear Internalization of Exogenous DNA into Mammalian Sperm Cells, Mol. Reprod. Devel., Vol. 34, pp. 133-139. Yet, certain inhibitory factors present in seminal fluid may inhibit this ability to take up DNA. Lavitrano, M., et. al. (1992) The Interaction Between Exogenous DNA and Sperm Cells, Mol. Reprod. Devel., Vol. 31, pp. 161-169. In addition, foreign DNA introduced into sperm cells may also suffer from extensive DNA rearrangement because in mature sperm cells, internalization of foreign DNA may activate certain endogenous nucleases in these cells. Maione, B. et. al. (1997) Activation of Endogenous Nucleases in Mature Sperm Cells upon Interaction with Exogenous DNA, DNA and Cell Biology, Vol. 16, pp. 1087-1097. Such rearrangement could threaten the usefulness of genetically modified animals using this technique.

Other work with sperm cells as vector have focused on the use of either lipid-based agents or electroporation to deliver foreign DNA into the sperm cells. Smith, supra; Rottman R., et. al. (1996) Liposome-mediated Gene Transfer via Sperm Cells. High Transfer Efficiency and Persistence of Transgenes by Use of Liposomes and Sperm Cells and a Murine Amplification Element, Journal of Animal Breeding and Genetics, Vol. 113, pp. 401-411; PCT Publications WO 99/42569, WO 99/40213, and WO 97/11597. Such methods may also suffer from the same problem of DNA internalization and exposure to nucleases that could cause rearrangement of the foreign DNA being introduced. In addition, lipid-based agents, which are often toxic, and electroporation may require extensive experimentation to prevent the death or the loss of sperm cell motility. Other techniques have also focused on using recombinant virus infection, as disclosed in PCT Publications WO 99/38991, or on using a “gene gun” with micro-carriers, as disclosed in PCT Publication WO 93/24626, to introduce foreign DNA into sperm cells. Such techniques may be technically challenging and may also affect the viability and motility of the sperm cells. They may also suffer from the same problem of DNA internalization and exposure to nucleases that could cause rearrangement of the foreign DNA being introduced.

Since 1989, researchers have reported the use of sperm cells as vectors in different animals ranging from insects, marine animals, amphibians, birds, and mammals. Smith, supra. However, few reported that the genetic modification was observed in viable mature offspring. Smith, supra. More problematic is the fact that some reports used only PCR analysis to verify the existence of the foreign DNA in the cells. These reports are summarized in table one of Gandolfi, F. (1998) Spermatozoa, DNA Binding and Transgenic Animals, Transgenic Research, Vol. 7, pp. 147-155. Since PCR cannot distinguish between foreign DNA transmitted through episomes or through the chromosomal DNA, Gandolfi has questioned the value of these reports stating that it “opens up an important argument relating to appropriate evaluation of the results described in some reports.” Gandolfi, supra. Episomal transmission is not as desirable as chromosomal transmission since the episome may be lost during subsequent cell division, and the desired effect of genetic modification may never be expressed in adult animals.

Because an easy, non-toxic, and efficient way of genetically modifying animals, especially in higher mammals, can greatly advance this field, a new way of using sperm cells for delivering genes into animals is needed.

### SUMMARY OF THE INVENTION

The present invention is directed to a vector and its use to introduce exogenous nucleic acid molecules and/or to generate genetically modified animals and cells. One aspect of this invention involves a vector that comprises a sperm cell and one or more polynucleotide molecules bound to a sperm cell through one or more non-liposome based linkers. The sperm cell can be any animal sperm cell, preferably non-human animal. In one preferred embodiment of this invention, the one or more polynucleotide molecules encode for a gene product that confers desired characteristics in the cells or the animals. In another preferred embodiment of this invention, the linker is a protein or polypeptide, preferably a sperm specific linker that binds with the external surface of the sperm cell. The linker interacts with one or more polynucleotide molecules preferably by ionic interaction. This interaction can also be carried out by different molecular interactions, including the use of another or secondary linker. The association of the sperm, linker, and the one or more polynucleotide can also occur in vitro or in vivo.

In another aspect of the present invention, genetically modified cells or animals are derived from the fertilization of an animal egg cell with the vector described above. Fertilization can occur in vitro or in vivo. In one preferred embodiment, genetic modification occurs with the polynucleotide molecule integrating, wholly or partially, into the cell or animal's genome. Another aspect of the present invention includes cells, such as sperm cells or egg cells, and cell lines that are derived from these genetically modified animals or their descendants.

In another aspect of the present invention, the genetically modified animals derived from the use of the sperm vector described above possess certain desired characteristics. Examples of these characteristics include faster growth rates, disease or pathogen resistance, high production of certain proteins in milk, and organs suitable for animal to human xenotransplantation.

In another aspect of the present invention, functional screening of polynucleotides can be achieved by introducing the polynucleotides into non-human animals. A kit or any other article of manufacture comprising the non-liposome linker may be provided, for example, together with instructions on how to introduce the polynucleotides into the animal by attaching polynucleotides to the sperm cell using the non-liposome linker. In another embodiment, a multi-well plate comprising the non-liposome linker and sperm cells from non-human animal may also be provided for high throughput functional screening of various polynucleotides. Various polynucleotides such as DNA encoding for various proteins or antisense RNA may then be attached to the sperm cells and introduced into animals, either by in vitro fertilization or artificial insemination, for determination of the function of the polynucleotide.

### BRIEF DESCRIPTION OF THE DRAWINGS

FIG. 1 is a pictorial representation of the basic steps involved in using one embodiment of the present invention.

FIG. 2 shows a flow-cytometry result of binding a sperm-specific antibody to mice's sperm cells as embodied in one aspect of the present invention.

FIG. 3 shows a flow-cytometry result of binding a sperm-specific antibody to pig's sperm cells as embodied in one aspect of the present invention.

FIG. 4 shows a flow-cytometry result of binding a sperm-specific antibody to cow's sperm cells as embodied in one aspect of the present invention.

FIG. 5 shows a flow-cytometry result of binding a sperm-specific antibody to chicken's sperm cells as embodied in one aspect of the present invention.

FIG. 6 shows a flow-cytometry result of binding a sperm-specific antibody to goat's sperm cells as embodied in one aspect of the present invention.

FIG. 7 shows a flow-cytometry result of binding a sperm-specific antibody to sheep's sperm cells as embodied in one aspect of the present invention.

FIG. 8 shows a plasmid map of pCMV-β.

FIG. 9 shows an agarose-gel analysis of a sperm-specific antibody binding to pCMV-β plasmid.

FIG. 10 show results of PCR analysis for the detection of pCMV-β sequences in genomic DNA isolated from mice's embryos genetically modified according to one embodiment of the present invention.

FIG. 11 shows results of southern-blot analysis for the detection of Hepatitis B surface-antigen gene-sequence in mice-tail-genomic DNA with this gene-sequence being integrated into the mice's chromosome according to one embodiment of the present invention.

FIG. 12 shows the plasmid map of pSEAP-2-control.

FIG. 13 shows the result of southern-blot analysis for the detection of pSEAP2-control plasmid sequence in the genomic DNA isolated from tail tissues of genetically modified pigs according to one embodiment of the present invention.

FIG. 14 shows the copy number of integrated pSEAP2-control plasmid in four genetically modified pigs based on densitometric intensities of bands in FIG. 13.

FIG. 15 and 16 show the results of enzyme assays for secreted alkaline phosphatase found in serum of pigs genetically modified according to one embodiment of the present invention.

FIG. 17 shows four F₁ generation mice, which have successfully received a pSEAP-2 DNA transgene transmitted from three F₀ generation founder mice, according to one embodiment of the present invention.

FIG. 18: panel (a) shows, in this experiment, that ten out of forty-three (23%) F₀ founder piglets chromosomal DNA have hybridization signals (3, 4, 25, 26, 33, 36, 38, 39, 40 and 42), indicating the presence of the pSEAP-2 exogenous DNA in these pigs; panel (b) shows, in this experiment, that nine F₁ generation piglets (17, 26, 36, 40, 42, 43, 44, 48 and 64) had a 1.3 kb positive hybridization band indicating the presence of the pSEAP-2 exogenous DNA in these pigs; panel (c) shows positive hybridization signals for F₁ generation pigs obtained from F₀ generation founder pigs # 4, 42, 8, 43, and 23, indicating the transmission of the pSEAP-2 exogenous DNA to the next generation.

FIG. 19 shows the results of enzyme assays for secreted alkaline phosphatase found in serum of a second group of pigs genetically modified according to one embodiment of the present invention.

FIG. 20: panel (a) shows the staining of the chromosomes from F₁ generation pig #152 using DAPI (4′,6′- diamindino-2-phenylindole Dihydrochloride), a DNA-specific fluorescent dye, panel (b) shows the same field of the chromosomes, but showing the hybridization signal of the FITC labeled probe fragment from pSEAP-2 hybridizing to chromosome 15, region q25-q28.

FIG. 21 shows the hybridization signal of chromsomal DNA from F₁ generation pigs bred from F₀ generation founder #31 where extra high molecular weight bands were observed in F₁ generation pig numbers 451, 452, 455, and 457.

FIG. 22: panel (a) shows the results of PCR analysis using ethidium bromide staining to detect the presence of a pGL-3 plasmid in F₀ generation chicken embryos; panel (b) shows the results of southern blot analysis of the same PCR products from panel (a) using an internal oligo probe hybridizing to sequences of pGL-3 plasmid.

FIG. 23 shows the expression of human interferon-β in F₀ generation chickens, wherein the y-axis shows the optical density reading at 450 nm, and the x-axis provides for the number assigned to the chicken.

FIG. 24 shows the result PCR analysis of ear and tail tissues from F₀ generation cows using ethidium bromide staining of the PCR product and hybridization by a ³²P end-labeled internal oligo to PCR products.

FIG. 25 shows the result of PCR analysis of ear and tail tissues from F₀ generation goats using hybridization by a 32P end-labeled internal oligo to PCR products.

FIG. 26 shows the map for plasmid pCMV-hIFbeta.

FIG. 27 shows F1 generation pigs, which have successfully received the pSEAP-2 plasmid transmitted from two F0 generation founder pigs, according to one embodiment of the present invention.

### GENERAL DESCRIPTION OF THE INVENTION

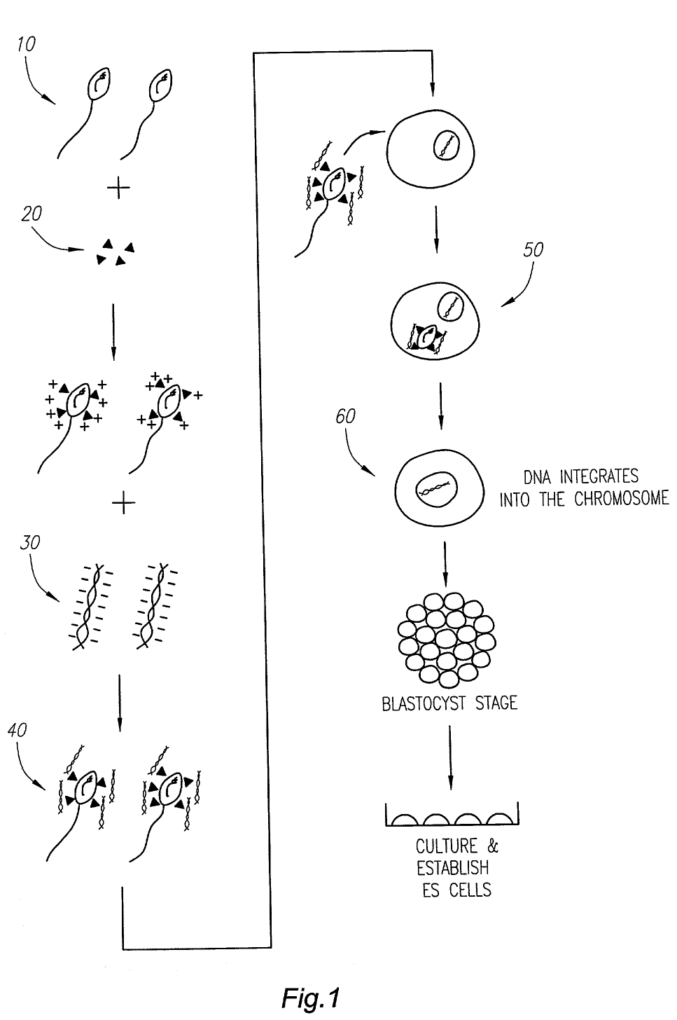

Generally, FIG. 1 shows the basic steps involved in using one embodiment of the present invention to introduce exogenous polynucleotides into animals or cells, or genetically modify cells or animals, using a sperm vector. Briefly, animal sperm cells 10, are collected by methods known in the art or purchased commercially from sources such as Birchwood Genetics in West Manchester, Ohio, and are bound together with linkers 20. These linkers are preferably antibodies or immunoglobulins of the types, IgG, IgA or IgM, but they can also be other compounds such as peptides, glycoproteins, carbohydrates, or other chemical-compound linkers. These linkers bind or associate to the sperm cells' external surface through different molecular interactions such as ionic interaction, covalent bonds, Vander Waals forces, or ligand-receptor interaction. Circular or linear DNA molecules 30 then bind or attach to the linkers on the sperm-linker complex also through different molecular interactions such as ionic, covalent bonds, Vander Waals forces, or ligand-receptor interaction. These DNA molecules may encode for certain gene products, but they may also be disrupted genes, homologous with endogenous genes, that recombine into the chromosome to knockout a gene. The sperm-linker-DNA complex 40 formed can then be used to effectuate fertilization in vitro or in vivo. Upon fertilization, the DNA is introduced into the fertilized egg 50 and embryo 60 and can integrate into the chromosome, becoming a part of an animal or cell's genetic material.

Alternatively, the binding, coupling, linking, attaching, or association of the sperm-linker-DNA complex can also be accomplished in vivo. The linker and the DNA can first be coupled or bound together in vitro. Afterwards, this linker-DNA complex can be injected directly or indirectly into a male animal's testicles. PCT Publications WO 99/40213 and WO 97/11597 disclose procedures for injecting DNA into the testicles, and these publications are incorporated herein by reference.

An example of a linker-DNA complex is an antibody attached with DNA molecules where the antibody specifically recognizes certain surface epitopes on sperm cells. Because of the acidic characteristic of naked DNA, it can ionically associate, bind or, couple with an antibody that has basic or positively charged properties. However, the DNA-linker interaction is not limited to ionic interaction. The complex can also be crosslinked by UV light to form covalent bonds by well known methods in the art. Both the DNA and the linker can also be modified by methods known in the art. For example, the DNA can be biotinylated by adding biotinylated deoxynucleotides in a PCR reaction; the antibody can be modified or purchased with attached streptavidin, which binds tightly to the biotin on the DNA; or a secondary antibody, which is modified with streptavidin and recognizes the first antibody can also act as a secondary linker between the modified DNA and the first linker.

If the DNA-linker complex is injected into the testis of the animal, this complex can seek out the sperm cells and bind to them. Fertilization can then occur in vivo via either natural copulation of the male and female animals or by artificial insemination of the female with collected sperm cells. The collected sperm cells can also be used with in vitro fertilization techniques, which are well known in the art. On the other hand, if binding of the sperm-linker-DNA complex, as a whole, occurred in vitro, fertilization can be achieved by in vitro fertilization techniques. The fertilized eggs and resulting embryos can then be transplanted to surrogate-animal mothers for development. Alternatively, well known artificial insemination methods or injections of the sperm-linker-DNA complex directly into the oviduct of female animals can also achieve fertilization in vivo.

Genetically modified animals can serve a number of useful applications. Livestock, poultry, or fish can be inserted with genes that encode for growth hormones to make them grow faster than normal or they can also be inserted with the somatotropin gene to increase muscle growth and decrease adipose tissue. Pursel, V. G., et.al. (1989) Genetic Engineering of Livestock, Science, Vol. 244, pp. 1281-1288; Etherton, T. D., et. al. (1993) Mechanism by which Somatotropin Decreases Adipose Tissue Growth, American Journal of Clinical Nutrition, Vol. 58 (Supp.), pp. 287S-295S. Inserting genes such as interferon that boost the immune system or other genes, such as genes encoding for viral, prion, or bacterial proteins, can also make these livestock, poultry, or fish disease or pathogen resistant. Examples of these infectious pathogens include Salmonella, influenza virus, prion proteins for the Mad Cow Disease, etc. Alternatively, introducing DNA encoding for anti-sense RNA molecules, which are complementary to these viral, prion, or bacterial RNAs, may also inhibit translation and production of proteins from these RNA, which limits growth and spread of these infectious pathogens.

Moreover, in animals, including insects such as silkworms, that produce raw materials for clothing such as wool and silk, inserting genes for biochemical enzymes that produce the rate-limiting amino acid may increase production of these raw materials. In sheep, for example, the availability of the amino-acid cysteine limits the production of wool. Inserting bacterial genes that encode for serine transacetylase and O-acetylserine sulfhydrylase may increase the conversion of serine and acetyl-CoA into cysteine, which in turn may increase production of wool. Ward, K., (1991) The Application of Transgenic Techniques for the Improvement of Domestic Animal Productivity, Current Opinion in Biotechnology, Vol. 2, pp. 834-839.

Furthermore, these genetically modified animals can also produce therapeutic proteins, such as insulin, growth hormone, interferon, erythropoietin, colony stimulating factor (GM-CSF), t-PA, or factor VIII, in their milk by joining the genes for these proteins with promoters from mammary specific genes such as sheep's β-lactoglobulin, mouse whey acid protein, or bovine αS1-casein. Id. On the other hand, the animal's milk can also be fortified with addition of humanized proteins, such as human lactoferrin that enhance the intestinal iron absorption in infants. Lonnerdal, B. (1996) Recombinant Human Milk Proteins—An Opportunity and a Challenge, American Journal of Clinical Nutrition, Vol. 63, pp. 622-626. Genetically modified pigs can even be a source for more “humanized” organs in animal to human xenotransplantation using genes such as human decay accelerating factor. Cozzi, E., et. al. (1994) Expression of Human Decay Accelerating Factor in Transgenics Pigs, Transplantation Proceedings, Vol. 26, pp. 1402-1403.

The articles cited above are all incorporated herein by reference.

The following examples demonstrate that the inventor has produced a number of genetically modified animals using the sperm vector as described above. Methods in molecular genetics, flow cytometry, antibody production, hybridoma technology, in vitro fertilization, embryo manipulation, and artificial insemination used but not explicitly described in this disclosure had already been amply reported in the scientific literature. These methods are well within the ability of one skilled in the art.

### EXAMPLE I

This example illustrates the preparation of an antibody specific to sperm cells.

Sperm cells collected from male mice were injected back into mice as antigens to immunize and produce antibodies reactive to sperm-surface antigens. Monoclonal antibodies, developed using common hybridoma techniques, were screened using flow cytometry to identify candidate antibodies that will bind to a series of different animals (mouse, pig, cow, sheep, goat, and chicken). Briefly, sperm cells were incubated with the different primary monoclonal antibodies, washed, and further incubated with a secondary antibody that specifically recognized mouse immunoglobulin. This secondary antibody, which was commercially available and well known in the art, had fluorescent molecules such as fluorescein or rhodamine conjugated to it. Once the secondary antibody molecules were bound and washed, the flow-cytometry instrument or the FACS sorter counted the number of fluorescent sperm cells with bound primary and secondary antibodies from naked sperm cells.

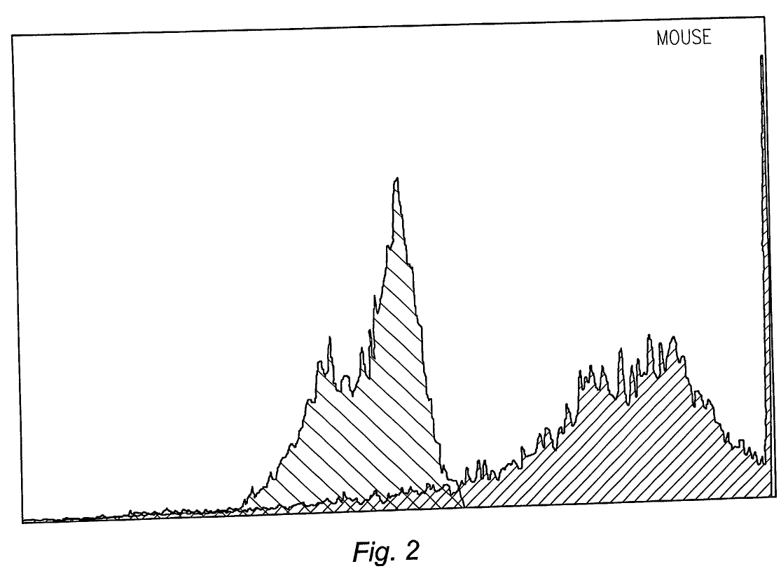

FIGS. 2-7 show these flow-cytometry analyses for mAbC that bind to sperm cells of mouse, pig, cow, chicken, goat, and sheep, respectively. The Y-axis corresponds to the number of sperm cells detected while the X-axis is the relative intensity of fluorescence bound to the cell. Cross-lined peaks denote control reactions where the sperm cells were incubated only with the fluorescent anti-mouse immunoglobulin antibody. On the other hand, the shaded peaks denote the reactions where mAbC antibody and the secondary antibody were incubated with corresponding sperm cells in a mouse, pig, cow, chicken, goat, and sheep, respectively. Right shifts in the peaks denote positive binding of the mAbC antibody.

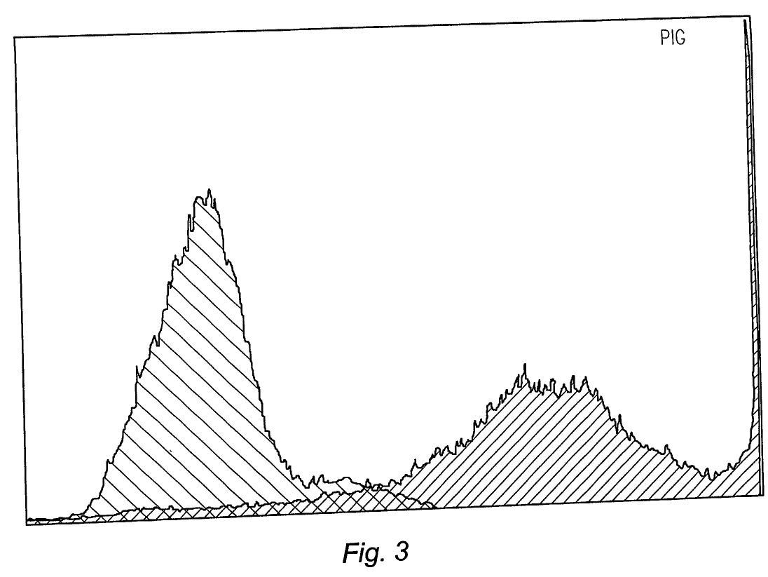

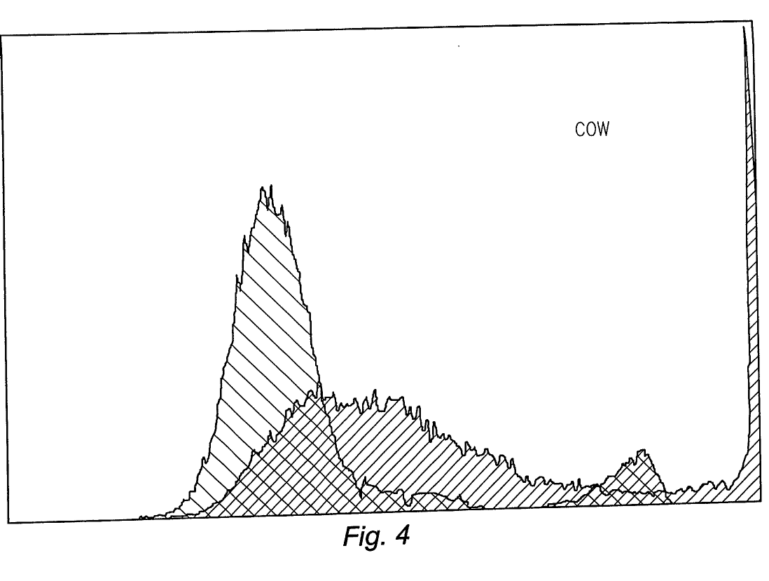

As can be seen in FIG. 2, greater fluorescence signals can be detected from mouse sperm cells incubated with mAbC and the fluorescent secondary antibody compared with sperm cells incubated with fluorescent secondary antibody alone. Similarly, in FIGS. 3 and 4, greater fluorescence can be detected from pig and cow sperm cells, respectively, incubated with mAbC and the fluorescent secondary antibody as evidenced by the right shaded peaks.

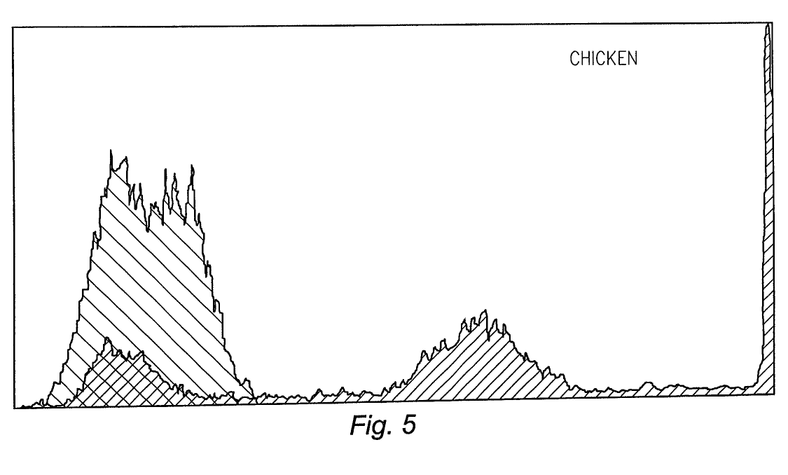

In FIG. 5, the incubation of the fluorescence antibody alone with the chicken sperm cells did not result in any fluorescence being detected in these sperm cells. In contrast, the right peak signified fluorescence in the chicken sperm cells that have attached mAbC antibodies. FIG. 5 also shows that some population of chicken sperm cells may not express the antigen recognized by mAbC as evidence by the left shaded peak.

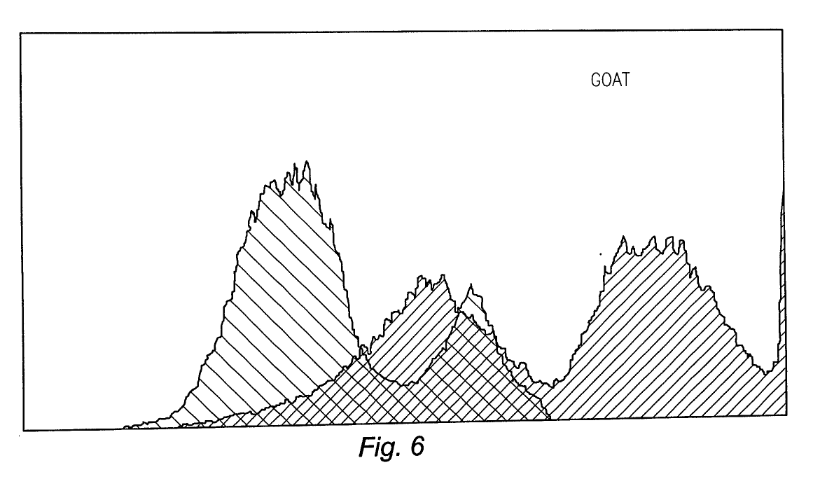

In FIG. 6, fluorescence can be detected from goat sperm cells incubated with mAbC and the fluorescent secondary antibody as evidenced by the two right shaded peaks. The left shaded peak may suggest a population of the goat sperm cells that express the antigen recognized by mAbC at a lower level than the population in the right peak. In contrast with the chicken sperm cells incubated with only the fluorescent secondary antibody in FIG. 5, the anti-mouse immunoglobulin fluorescent antibody seems to also bind to the goat sperm cells, but at a much reduced level than with mAbC acting as a linker.

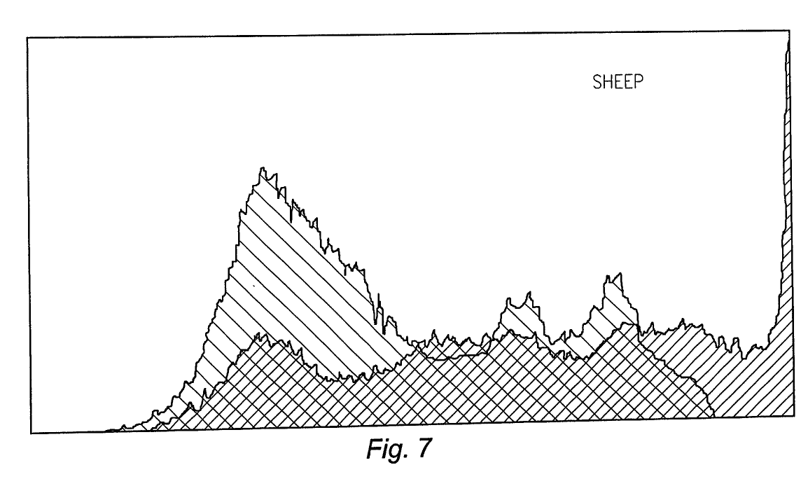

Similarly, in FIG. 7, fluorescence can be detected from sheep sperm cells incubated with mAbC and the fluorescent secondary antibody as evidenced by the right shaded peaks. The distribution of the peaks again suggests the possibility that different sperm cells have different levels of the antigen recognized by mAbC.

As seen in FIGS. 2, 3, 4, 6, and 7, mammalian sperm cells bind, at some lower level, to the fluorescent secondary antibody. Since the secondary antibody is directed to a mouse immunoglobulin, it may have cross reactivity to other mammalian proteins on the sperm cell surfaces, which are not present in the chicken sperm cells (FIG. 5). Nevertheless, the shifts in fluorescence peaks upon addition of mAbC clearly demonstrate the higher affinity of the mAbC antibody to different animal sperm cells.

### EXAMPLE II

This example illustrates the ability of the monoclonal antibody mAbC to bind to DNA molecules through ionic interaction.

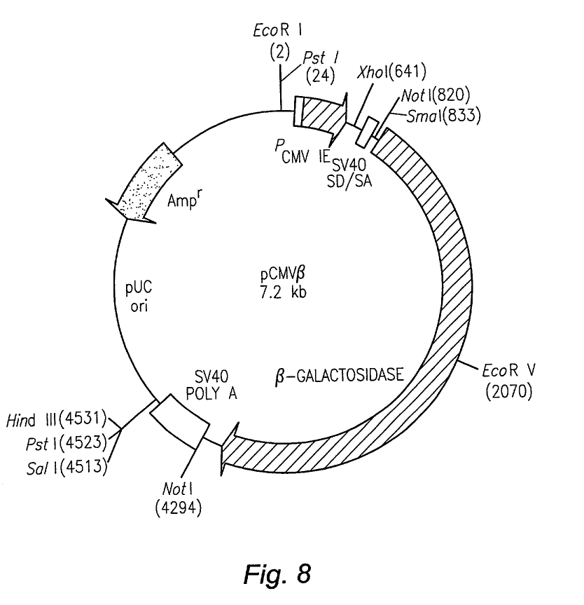

Different volumes of purified solutions of mAbC at a concentration of 0.5 mg/ml were added to DNA solutions containing 300 ng of Sal I cut pCMV-β plasmid (FIG. 8, Clontech Laboratories, Inc., Cat. # 6177-1). After incubating the mixtures at room temperature for forty minutes, the mixtures were loaded on a regular one percent agarose gel and run at 20 milli-amps for one hour. Afterwards, the DNA was stained with Ethidium Bromide and visualized under UV light.

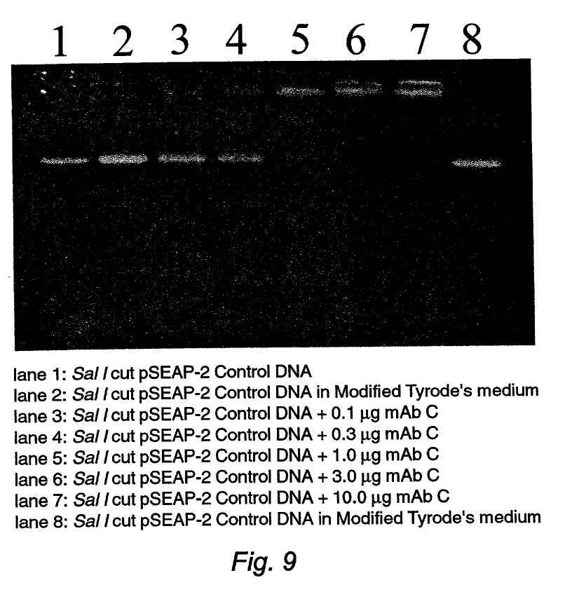

In FIG. 9, lanes 1, 2, and 8 were controls with lane 1 being pure Sal I cut pCMV-β plasmid and lanes 2 and 8 being Sal I cut pCMV-β plasmid in Modified Tyrode's medium. Lanes 3, 4, 5, 6, and 7 corresponded to experimental reactions with the Sal I cut pCMV-β plasmid incubated with 0.2 μl, 1 μl, 2.5 μl, 6 μl, and 10 μl of mAbC at 0.5 mg/ml. In lanes 5, 6, and 7, increasing amounts of DNA were retained in the wells of the gel, showing that association of the antibody, which has a positive charge, with the plasmid DNA, which has a negative charge, yielded a net electric charge of zero, resulting in a complex that no longer responds to the electric field in the gel.

### EXAMPLE III

This example illustrates the binding or coupling of the DNA to the sperm via the linker or antibody.

DNA molecules, labeled with P³² using standard end labeling techniques with T4 DNA polymerase, were incubated with mouse, pig, chicken, sheep, goat, and cow sperm cells together with either mAbC, mAbD, or a control antibody specific to a Drosophila protein. The amount of DNA binding was measured by scintillation counting. The ratio of sperm cells to antibody were as follows:

Mouse—400 thousand sperm cells to 600 ng of labeled DNA;

Pig—600 thousand sperm cells to 800 ng labeled DNA;

Chicken—300 thousand sperm cells to 500 ng of labeled DNA;

Sheep—1million sperm cells to 500 ng of labeled DNA;

Goat—1million sperm cells to 500 ng of labeled DNA; and

Cow—1 million sperm cells to 500 ng of labeled DNA.

Table 1 shows that with the presence of mAbC and mAbD, sperm cells significantly bound more labeled DNA compared with reactions with no antibody or with the Drosophila protein-specific antibody. Reactions 1 and 2 contained only sperm cells and labeled DNA, while reactions 3 and 4 contained the Drosophila-protein-specific antibody together with sperm cells and labeled DNA. Reactions 5 contained mAbD while reactions 6 and 7 contained mAbC together with sperm cells and labeled DNA.

TABLE 1

| Reac- | | Mouse | Pig | Chicken | Sheep | Goat | Cow |
|---|---|---|---|---|---|---|---|
| tions | | (cpm) | (cpm) | (cpm) | (cpm) | (cpm) | (cpm) |
| 1 | no | 12471 | 12971 | 5830 | 15367 | 17749 | 12766 |
| | anti- | | | | | | |
| | body | | | | | | |
| 2 | no | 15814 | 13713 | 6383 | 13259 | 16574 | 14398 |
| | anti- | | | | | | |
| | body | | | | | | |
| 3 | Control | 11541 | 10531 | N/D | 14018 | 155347 | 15351 |
| | Anti- | | | | | | |
| | body | | | | | | |
| 4 | Control | 13653 | 14038 | N/D | 12834 | 15997 | 13918 |
| | Anti- | | | | | | |
| | body | | | | | | |
| 5 | mAbD | 18900 | 16220 | 10314 | N/D | N/D | N/D |
| 6 | mAbC | 18139 | 16269 | 7294 | 19368 | 20385 | 20417 |
| 7 | mAbC | 19314 | 17343 | 9865 | 18437 | 19543 | 18643 |

N/D = Not determined

### EXAMPLE IV

This example illustrates the procedures carried out to introduce exogenous nucleic acid molecules and/or to generate genetically modified mice.

Sperm cells were collected from dissected epididymis of nine to twenty weeks old FVB male mice. Cut into small pieces, these epididymis tissues were incubated in 300 μl of Modified Tyrode's medium at pH 7˜8 for one hour to allow the sperm cells to escape into the medium. Once the sperm cells were collected in 300 μl of medium, five micrograms of the linker antibody were added to one million sperm cells at 37° C. for one hour. The sperm-linker complex was washed three times with 300 μl of Modified Tyrode's medium using a standard microcentrifuge set at 3000 rpm for one and a half minutes. The sperm-linker complex was finally resuspended in 300 μl of medium, and one microgram of linearized pCMV-β plasmid, a plasmid encoding for Hepatitis B surface antigen (HBsAg), or pSEAP-2 plasmid was added and incubated for one hour.

To collect ovulated eggs, nine to twelve weeks FVB female mice each received an injection of 5 I.U. Pregnant Mares Serum (PMS). After about 48 hours, each mice also received an injection of 5 I.U. of human chorionic gonadotropin (hCG). About 12-14 hours later, the mice were sacrificed and their ovulated eggs were dissected. Dissected ovulated eggs surrounded by cumulus cells were placed in a 35-mm petri dish containing a drop of Modified Tyrode's medium at room temperature. Afterwards, 300 μl of sperm-linker-DNA complex prepared as described above were added directly to the ovulated eggs. The whole mix was equilibrated with CO₂ at 37° C. with mineral oil added on top to prevent evaporation. After four hours of in vitro fertilization at 37° C., fertilized eggs were collected with capillary tubes and washed thrice with CZB medium. The embryos were further incubated in 300 μl of CZB medium for 20-22 hrs before being transferred to oviducts of pseudo-pregnant female mice.

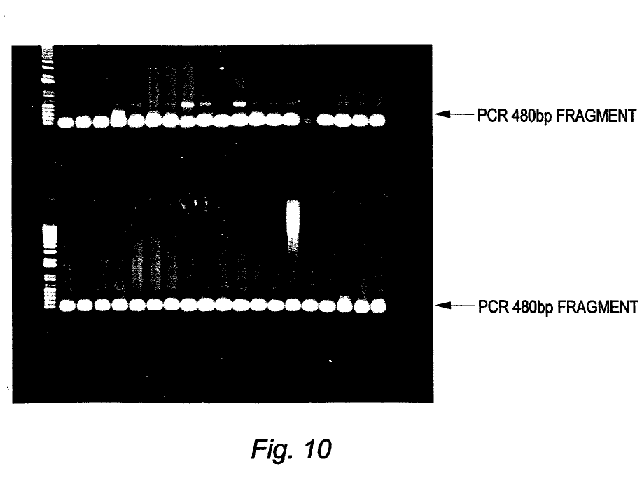

To confirm the presence of the pCMV-β plasmid, genomic DNA isolated from embryos, ten days after transplantation into the pseudo-pregnant female mice, were analyzed by PCR using primers that detect a 480 bp fragment corresponding to the CMV promoter region of the pCMV-β plasmid (FIG. 8). Genomic DNA was isolated from 10-day-old mouse embryos using the standard phenolchloroform extraction protocol. 100 ng of genomic DNA was used for PCR in a 50 μl reaction using Taq DNA polymerase (Promega). The primer pair were 5′-AGTACATCAATGGGCGTGGATAGCGGTT-3′(forward bp 347-374) and 5′-TTGCGGCCGCGGGTACAATTCCGCAGCT-3′(backward bp 799-827) in the CMV promoter linker regions of pCMVβ plasmid DNA. The amplification was carried out according to the following conditions: 1 min at 94° C. followed by 35 cycles at 94° C. for 1 min, then 67° C. min and 72° C. for 3 min with a final extension step at 72° C. for 5 min. PCR samples were analyzed on a 1.2% agarose gel. In FIG. 10, lanes 6, 7, 8, 9, 10, 12, 13, 14, 15, 17, 18, 19, 24, 33, and 40 clearly show this 480 bp PCR fragment. Lanes 1 and 21 corresponded to the molecular size markers.

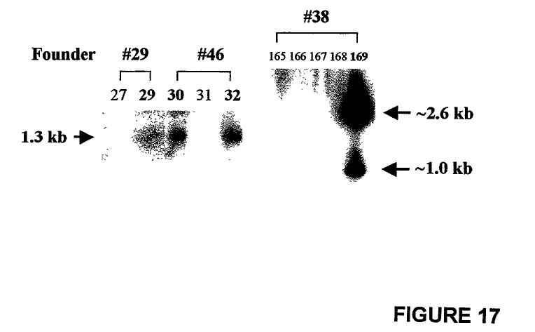

In another experiment with pSEAP-2 plasmid, forty-seven F₀ pups were born but no hybridization signal was detected in their tail samples. However, four (33%) transmitted transgenic mouse lines (F₁) were found after twelve F₀ founders were randomly selected to mate with the wild type mice. A 1.3 kb fragment was detected by Southern blot analysis in one out of ten offspring from founder 29; two out of three offspring from founder 46; and one out of nine offspring from founder 33 (not shown) (FIG. 17). We also noticed that one out of five offspring from founder 38 showed multiple hybridization bands (about 1 and 2.6 kb) indicating DNA rearrangement (FIG. 17). This is a higher frequency of mosaicism than most published results in FVB/N mice produced by the microinjection method. The fertilization rate may be improved and the method simplified by using alternatives to in vitro fertilization such as oviduct, uterine, or artificial insemination. Since mAbC bound to all tested sperm from various species, the disclosed method could be applicable with different strains of mice as well as in other rodents such as the rat.

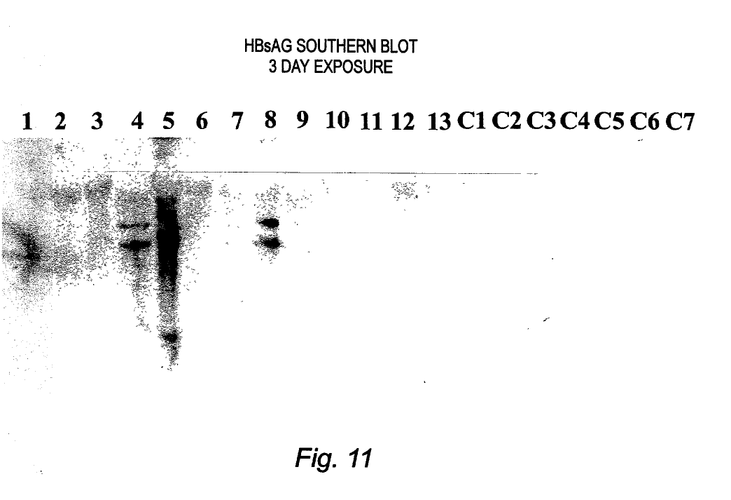

To confirm integration of the HBsAg plasmid into the mice genome, southern blot analysis were also performed. Genomic DNA isolated from mice's tails were digested, ran on a gel, transferred to a nylon membrane according to methods known in the art. FIG. 11 shows the southern blot hybridization results with complementary probe sequences to HBsAg. Lanes 1-13 contained genomic DNA from mice born from pseudo-pregnant mice that received embryos fertilized with the sperm-linker-DNA complex described above; lanes C1-C7 contained genomic DNA from mice that were untreated or non-transgenic mice. Lanes 4, 5, and 8 show bands positive for HBsAg sequences integrated in the mice's genome, thus, demonstrating that three out the thirteen mice were genetically modified.

### EXAMPLE V

This example illustrates the procedures carried out to introduce exogenous nucleic acid molecules and/or to generate genetically modified pigs.

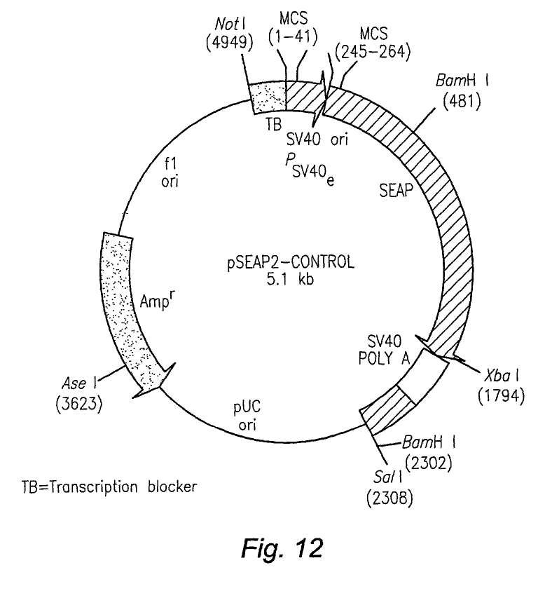

Ejaculated sperm cells from pigs were collected using methods generally known in the art of animal husbandry. Suspended in one milliliter of pig extender medium (purchased from Merck, Germany, Ref.N.R.13515/0001—dilute mixture M3 for boar sperm), fifteen million sperm cells were incubated with five micrograms of the linker antibody for forty minutes at room temperature with intermittent shaking in between. After washing the sperm-linker mixture once with pig extender medium and finally resuspending the mixture in 1.5 ml of the same medium, five micrograms of the plasmid pSEAP2-control (FIG. 12, Clontech Laboratories, Inc., Cat. # 6052-1) were added and incubated with the mixture for forty minutes at room temperature. Direct injections of 200 μl of the resulting sperm-linker-DNA complex into the oviducts of anesthetized female pigs resulted in fertilization in vivo.

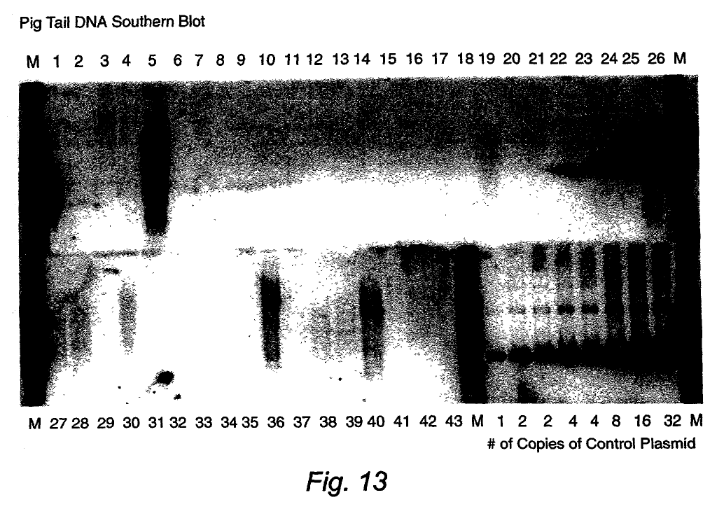

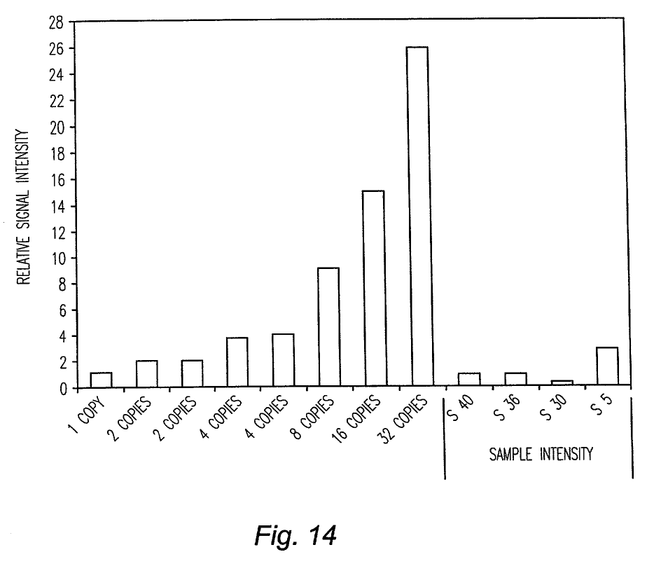

After the pigs were born (F₀ generation) and grown to 70-day-old pigs, they were analyzed for the presence of the pSEAP2-control plasmid. FIG. 13 shows the southern blot analysis of genomic DNA isolated from the tail tissues of these pigs. Briefly, genomic DNA isolated from these pigs were digested, run on a gel, and transferred to a nylon membrane according to methods well known in the art. The blot was then probed with labeled sequences from the Not I to BamH I region of the pSEAP2-control plasmid shown in FIG. 12. In FIG. 13, M denotes the marker lanes, and 1-43 denotes the number of pigs analyzed. Hybridization signals in lanes 5, 17, 19, 25, 26, 27, 28, 30, 36, 38, 39, and 40 indicated that the pSEAP2-control plasmid had integrated into the corresponding pig's genome. In the lower right half of the figure, eight lanes with increasing copies of pSEAP2-control plasmid molecules (1, 2, 2, 4, 4, 8, 16, and 32) were also loaded on the gel together with the DNA from the experimental pigs. These eight lanes were used to estimate the copy number of pSEAP2-control plasmid integrated into the pigs based on the densitometric intensities of the bands (FIG. 14). As can be seen in FIG. 14, S5 had the highest intensity, which corresponds to lane 5 of FIG. 13.

Further breeding of F0 generation pigs #25 and #38 produced F₁ generation pigs that also have the pSEAP-2 control plasmid integrated into the genome. Southern blots were used to detect the presence of the pSEAP-2 plasmid in F₁ generation pigs. Briefly, extracted genomic DNA from pig tails were digested with Bgl I, which will produce a 1.3 kb band because of two internal Bgl I restriction sites in the pSEAP-2 control DNA. A radioactive probe was generated by random priming using the same 1.3 kb Bgl I DNA fragment (bp 3570-4838) digested from the plasmid pSEAP-2, as template.

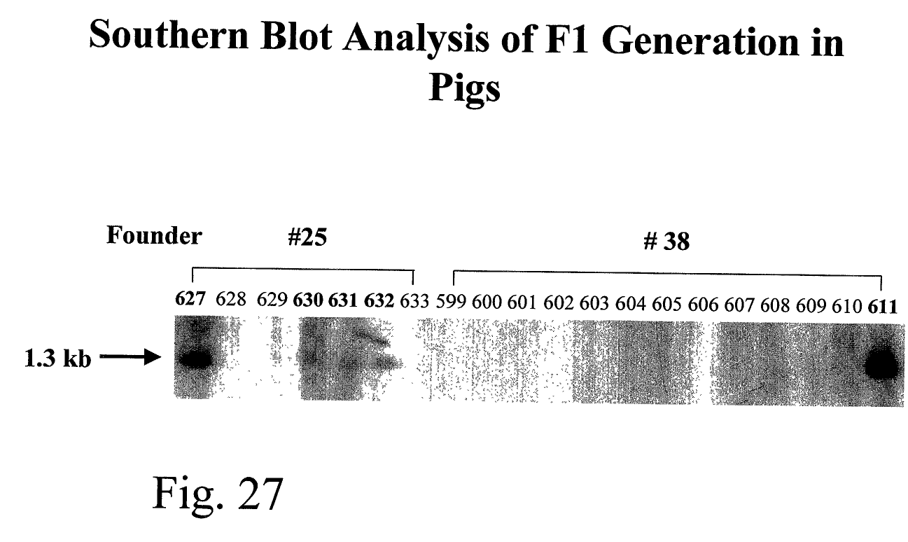

Initial analysis of three F₁ generation offsprings from pig #25 did not indicate a positive result with Southern blot analysis. Further analysis of greater number of F₁ generation pigs from F₀ pig #25, however, showed that, in fact, four out of seven (4/7) F₁ pigs analyzed were positive for the pSEAP-2 plasmid. FIG. 27 shows the result of this latter analysis wherein F₁ pigs 627 and 630-632, showed positive for the 1.3 kb band. Similarly, pig number 611 from founder F₀ pig 38 also showed a positive 1.3kb band. As seen from FIG. 27, the pSEAP-2 plasmid introduced into the F0 generation was successfully transmitted through the germ line and into the progeny of founder pig #25 and pig #38.

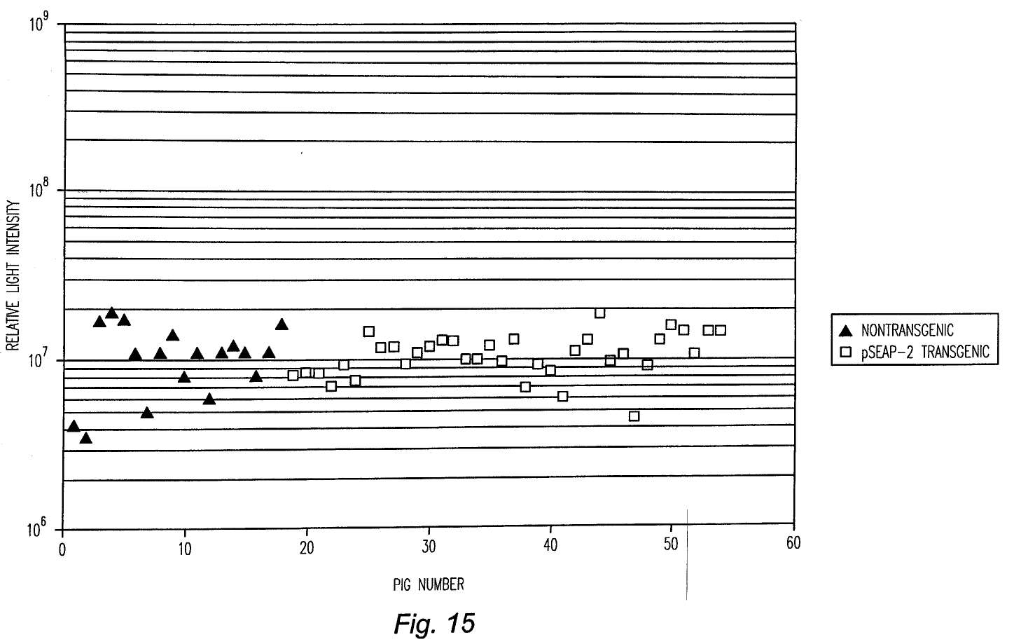

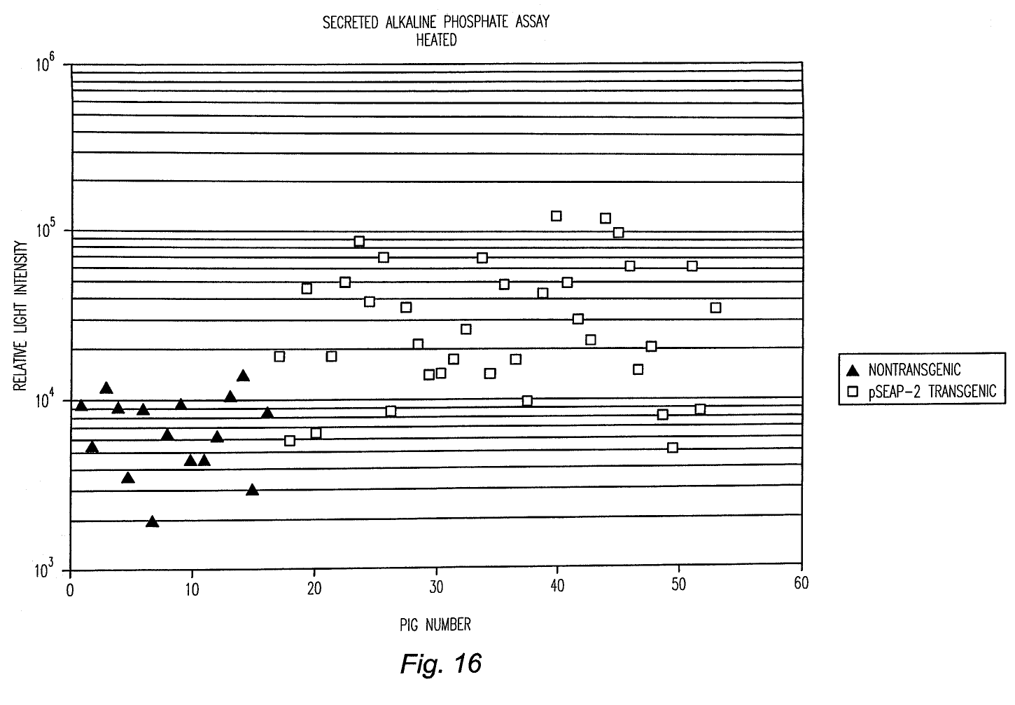

In another study, secreted alkaline phosphatase (SEAP) expressed from the pSEAP2-control plasmid was also detected in the 43 F₀ generation pigs. When the pigs were 70-day old, serum from these pigs were collected and assayed for SEAP activity using Clontech's Great EscAPE™ SEAP Chemiluminescence Detection Kit (Cat. # K2041-1) and its protocol, which is incorporated herein by reference. The SEAP enzyme expressed from Clontech's pSEAP-2 vector is thermostable. Thus, to determine the level of SEAP activity as opposed to the pigs' endogenous alkaline phosphatase enzyme activity, the assay required the deactivation of the endogenous alkaline phosphatase enzyme by heating the samples at 65° C. for thirty minutes before adding the chemiluminescence substrate. As a control, FIG. 15 shows the result of the assay without performing this heat deactivation step. The level of total alkaline phosphatase activity was not significantly different between the genetically modified pigs and non-transgenic control pigs. In contrast, FIG. 16 shows the result including this heat deactivation step. Without the endogenous alkaline phosphatase activity, SEAP activity was significantly higher in the genetically modified pigs than in the non-transgenic control pigs. A reading of more than 2×10⁴ RLU of SEAP activity was set as positive in the genetically modified pigs, which is significantly higher than in the nontransgenic controls (average 6.9×10³±3.7×10³ RLU). Based on this level of SEAP activity, twenty-one out of the thirty six founder pigs (58%) were shown to express the thermostable human SEAP enzyme. Due to the mosaic condition in many of the transgenic pigs, the number of (58%) founders in the first experiment expressing human SEAP in their sera is more than the number of founders found to be positive (12/43, ˜30%) at least once by Southern blot analysis.

### EXAMPLE VI

This example further illustrates the procedures used to introduce exogenous DNA and/or to generate genetically modified pigs.

Female pigs (gilts) between 10 and 14 months old of three different breeds were selected: Duroc, Yorkshire, and Landrace. They were divided in two groups: 27 in group I and 30 in group II. Ovulating pigs were sequentially anesthetized and surgically inseminated by injecting 0.2 ml of sperm (about 1.2×10⁶)/mAb C/Sal I-linearized pSEAP-2 control DNA mix, as prepared in Example V, into each side of the oviduct. Forty-three offspring from 7 gilts in group I (total 27 gilts were inseminated with a 26% pregnancy rate) were obtained. These forty-three offspring (F₀ generation) are the same animals described in Example V. On the other hand, group II pigs produced a total of thirty-two offsprings (F₀ generation) from 8 gilts (total 30 gilts inseminated with a 28% pregnancy rate).

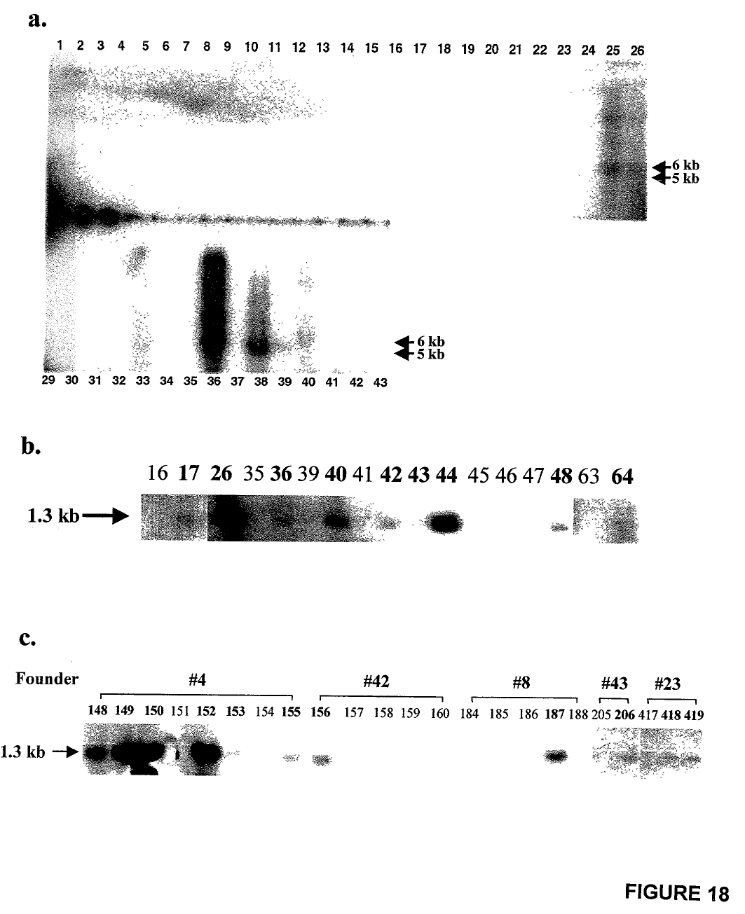

In a different experiment analysis than the one conducted in Example V, tail-tip DNA from 2-7 day old piglets (F₀ founders) was isolated and digested with EcoRI, which is a unique restriction site in the pSEAP-2 control DNA. To avoid cross hybridization with the endogenous alkaline phosphatase gene, a Not I/Sal I fragment in the plasmid vector region was labeled as a probe for Southern blot analysis. FIG. 18a shows, in this experiment, that ten out of forty-three (23%) F₁ founder piglets have hybridization signals (3, 4, 25, 26, 33, 36, 38, 39, 40 and 42), indicating the integration of the pSEAP-2 control gene into the pig genome. Piglets 4, 33, 36, 38 and 39 show a 5 kb band suggesting the integration of multiple copies of the foreign gene into the pig genome with a head to tail linkage while numbers 3, 25, 26, 40 and 42 show a 6 kb band most likely caused by integration with a tail to tail linkage tandem repeat. The low intensity of hybridization bands from a large portion of positive samples suggests a low-copy number of the transgene.

To ensure detection of all transgenic samples including a single copy of the transgene, genomic DNA from the mid-tail region of the piglets was also examined by digesting the extracted DNA with Bgl I digestion. FIG. 18b shows, in this experiment, that ten out of seventy-five F₀ founder piglets (forty-three from group I and thirty-two from group II; 5 (not shown), 17, 26, 36, 40, 42, 43, 44, 48 and 64) had a 1.3 kb positive hybridization band.

Compared with the Southern blot analysis using EcoR I digestion on the tip region, only numbers 26, 36, 40 and 42 showed positive signals for both tail regions. Furthermore, analysis of sperm genomic DNA from a limited number of 8-month-old F₀ founders (boars) showed that two out of eleven (18%), numbers 25 and 30, had a hybridization signal (data not shown). These results indicate that many founders of transgenic pigs are mosaic, that is, some cells in the body actually carries the pSEAP-2 DNA, but some do not. Thus, the detection of the transgene is dependent on the relative ratio of cells with or without the transgene in a given tissue.

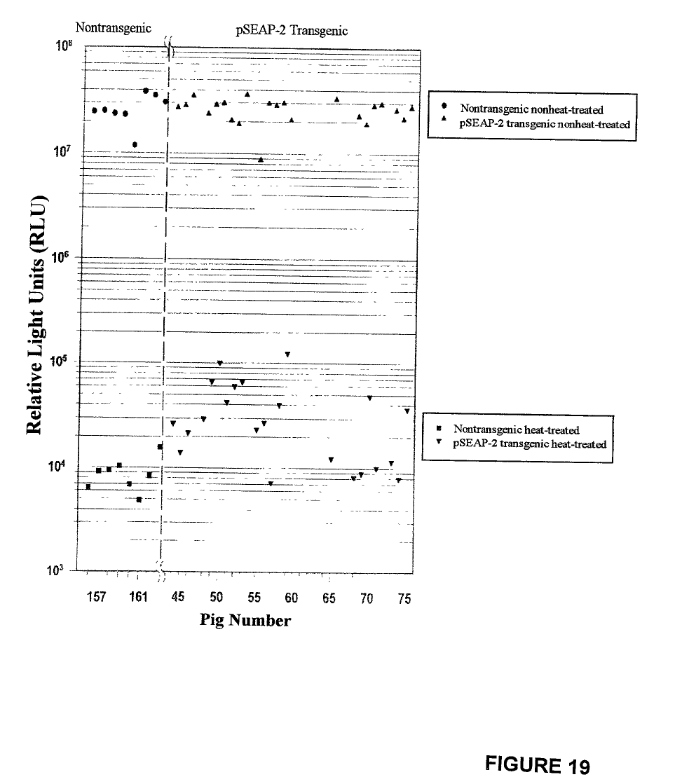

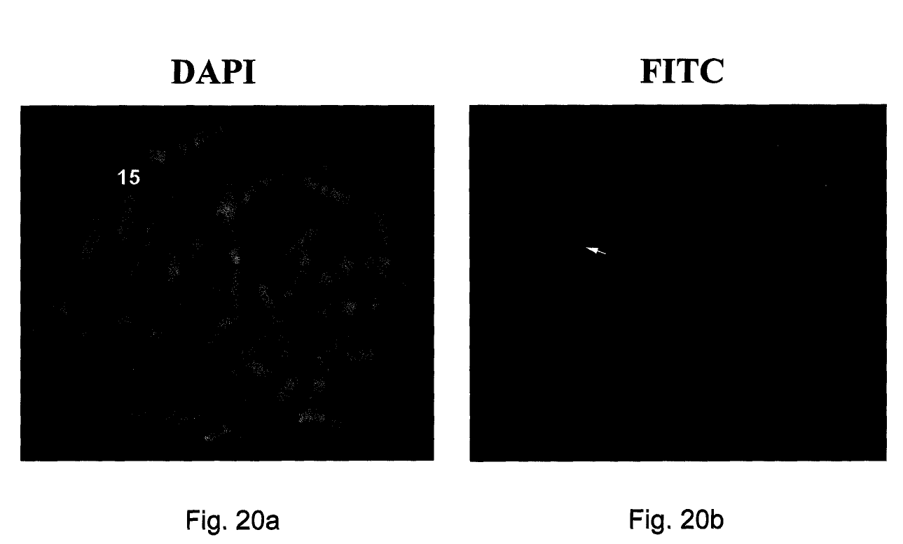

As an alternative way to detect transgenic pigs, detection of the expression of heat stable SEAP in the sera collected from the 70-day-old founders, as well as those from the 90-day-old founders from group II (samples #44-75) was performed. Briefly, fifteen μls of heat-treated and nonheat-treated sera from each founder were assayed for chemiluminescence in triplicate. The results for the group I pigs are described in Example V above. With respect to group II, the sera from the second group of founders were assayed and showed similar results: fourteen out of twenty-two (64%) founder pigs showed comparable levels of expressed SEAP (>2×10⁴ RLU) (FIG. 19).

The amount of expressed human SEAP in the transgenic pigs' sera was also estimated using a standard curve. We selected founders with a human SEAP level between 2 x 104 RLU to 1.2×10⁵ RLU as samples. About 18-112 ng of expressed SEAP was found in 0.25 ml of serum from 70-day-old pigs. The average weight of the 70-day-old pigs was 27.2 kg. If pig weight (kg) to pig blood volume (liter) is 13:1 and the ratio of blood volume to serum volume is 2:1, then it can be estimated that the transgenic pigs expressed 72.3- 450.2 μg of human SEAP per pig. Since the animals were mosaic, the potential transgene expression may be even higher.

### EXAMPLE VII

A study was also conducted using fluorescent in-situ hybridization (FISH) analysis to further evidence that the transgene pSEAP-2 successfully integrated into the chromosome and was transmitted to F, generation.

A 3.1 kb EcoR I/ Sal I DNA fragment from the plasmid vector region of the pSEAP-2 control plasmid was used as a probe by labeling the fragment with fluorescein isothiocyanate (FITC). Tissue samples from F₁ pig #152, bred from F₀ founder pig #4 and the fragment were provided to SeeDNA Biotech, Inc. (Windsor, Ontario, Canada), who provided FISH analysis service using standard FISH protocols. Briefly, lympocytes were isolated from the pig spleen and cultured at 37° C. in RPMI 1640 medium supplemented with 15% fetal calf serum and phytohemagglutinin (PHA). After 44 hours, the cultured lymphocytes were treated with 0.18 mg/ml BrdU for an additional 17 hours. The synchronized cells were washed and recultured at 37° C. for 6 hours in α-MEM with thymidine (2.5 μg/ml). Chromosomes slides were made by conventional method as used for human chromosome preparation (hypotonic treatment, fixation and air dry).

DNA probe was biotinylated with dATP using Gibco BRL BioNick Labeling kit. The procedure for FISH detection was performed according to Heng et al, High resolution mapping of mammalian genes by in situ hybridization to free chromatin, Proc. Natl. Acad. Sci. USA 89:9509-9513 (1992) and Heng and Tsui, Modes of DAPI banding and simultaneous in situ hybridization, Chromsoma, 102: 325-332 (1993), which are hereby incorporated by reference. Briefly, slides were baked at 55° C. for one hour. After RNase A treatment, the slides were denatured in 70% formamide in 2×SSC for 2 minutes at 70° C. followed by dehydrated with ethanol. Probes were denatured at 75° C. for 5 minutes in a hybridization mix consisting of 50% formamide and 10% dextran sulphate and pre-hybridized for 15 minutes at 37° C. Probes were loaded on the denatured slides. After overnight hybridization, slides were washed and detected as well as amplified using published method (Heng et. al., supra). FISH signals and the DAPI banding pattern were recorded separately by taking photographs, and the assignment of the FISH mapping data with chromosomal bands was achieved by superimposing FISH signals with DAPI banded chromosomes.

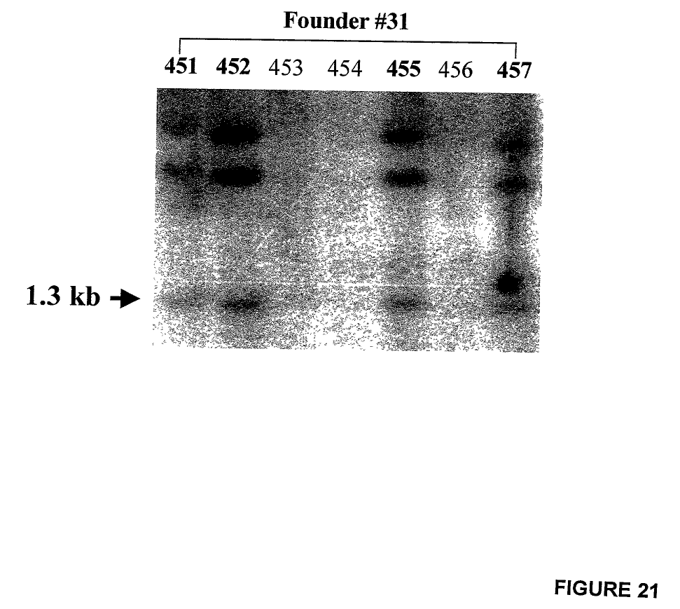

FIG. 21a shows the staining of the chromosomes from F₁ pig #152 using DAPI (4′,6′-diamindino-2-phenylindole Dihydrochloride), a DNA-specific fluorescent dye. FIG. 21b shows the same field of the chromosomes, but showing the hybridization signal of the FITC labeled probe fragment from pSEAP-2. Under the condition used, there was one locus detected from FISH. The detailed localization of the pSEAP-2 integrated into the chromosome was determined based on the summary from 10 photos, and based on the DAPI banding, the pSEAP-2 DNA was mapped to chromosome 15, region q25-q28.

Thus, the results of the FISH analysis evidence not only the integration of the pSEAP-2 control plasmid into the chromosome of the F₀ founder pig #4, but also that it has integrated into the germline of pig #4 allowing the transmission of the pSEAP-2 control plasmid to the next generation's (F₁ generation pig #152) chromosome.

### EXAMPLE VIII

This example further illustrates the transmission of the transgene through the germline of F₀ generation pigs and to F₁ generation pigs.

In one experiment, sixteen F₀ founders described in Examples V and VI were randomly selected to mate with wild type pigs to demonstrate transmission of the germ-line. The F₁ generation from six (37.5%) founders were found to carry a 1.3 kb positive hybridization band (FIG. 18c): seven out of sixteen offspring from founder 4 (male Duroc); one out of five offspring from founder 8 (male Yorkshire); two out of fifteen offspring from founder 23 (female Yorkshire); seven out of thirty offspring from founder 31 (male Yorkshire); one out of five offspring from founder 42 (female Landrace), and one out of nine offspring from founder 43 (female Landrace). The described method using the sperm-linker DNA mixture was successful in producing transgenic pigs of different strains without gender selection.

Interestingly, the offspring of founder 4, numbers 149, 150 and 152, had the strongest hybridization intensity, followed by number 148 and then 153 and 155, while 151 and 154 showed no signal (FIG. 18c). This may be an indication of multiple site transgene integration. Also observed was extra high molecular weight bands in the offspring of founder 31: numbers 451, 452, 455, and 457 (FIG. 21). DNA rearrangement has probably occurred in the case for founder #31. Different intensities of hybridization signals on the same blot were seen in number 152 from founder 4, number 156 from founder 42, and number 187 from founder 8 (FIG. 18c).

This may suggest that different copies of the transgene have integrated into the various pig genomes.

These data suggest that a high germ-line transmission rate, multiple site insertion, rearrangement, and multiple DNA-copy insertion may be obtained by the disclosed method. In addition, founders 4, 31, 42, and 43 were able to transmit the transgene to some of their offsprings as determined by Southern blot. Although founders 8 and 23 didn't show any detectable hybridization signal, some of their offsprings were positive for the transgene. This indicates that some founders of transgenic pigs generated by the method may be mosaic but still have germ-line transmission capability. The mosaicism observed may also be due to transgene integration at various stages during early embryonic development.

### EXAMPLE IX

This example illustrates the introduction of exogenous DNA into and/or the genetic modification of chickens.

Fresh semen from 50-week-old Leghorn roosters was collected, and the sperm content was adjusted to 2×10⁸ spermatozoa in 1.5 ml extender (Lago). 10 μg of mAb C. was added to the sperm mixture and incubated for 1 hour at room temperature with occasional mixing. The sperm mixture was then equally distributed into 5 microcentrifuge tubes, spun at 3,000 rpm for 3 minutes, and the supernatant was removed. The sperm pellet was washed once with 200 μl of incubation buffer (extender+1.5 mg/ml BSA) and each pellet was resuspended in 300 μl incubation buffer and pooled together. 10 μg of Sal I linearized pGL-3 control DNA (Promega, #E1741) was added and the mixture was incubated for 1 hour at room temperature with occasional mixing. Finally, 1.7 ml of incubation buffer was added. Artificial insemination was performed on 20 hens (each with 10⁷ sperm in 0.1 ml).

After artificial insemination, 7-day-old chicken embryos were harvested for PCR analysis. A small piece of 7-day-old chicken embryo tissue was digested in 100 μl PCR buffer with 1 mg/ml proteinase K, 0.45% NP 40 and Tween 20 at 55° C. overnight. 2 μl of the sample was used for PCR in a 25 μl reaction using RedTaq DNA polymerase (Sigma). The PCR primers used were 5′-TTACAGATGCACATATCGAG-3′ (forward bp 404-422) and 5′-CATACTGTTGAGCAATTCAC-3′ (reverse bp 614-632), both in the luciferase gene region of the pGL-3 control plasmid (Promega). The amplification was carried as follows: 4 min at 95° C. followed by 30 cycles at 53° C. for 1 min, 72° C. for 1 min and 94° C. for 1 min, followed by 1 cycle at 53° C. for 1 min and 72° C. for 10 min. PCR samples were analyzed on a 1.5% agarose gel and transferred to a nylon membrane. A ³²P-end labeled third internal oligo (5′-GGTGGACATCACTTACGCTGAGTA-3′, bp 423-446) was also used to verify the PCR product by hybridization.

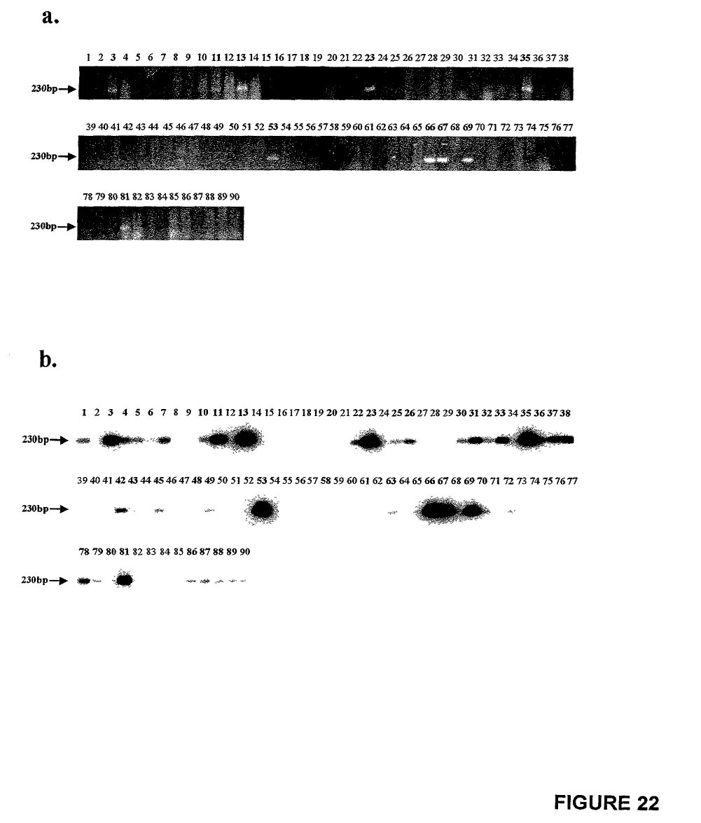

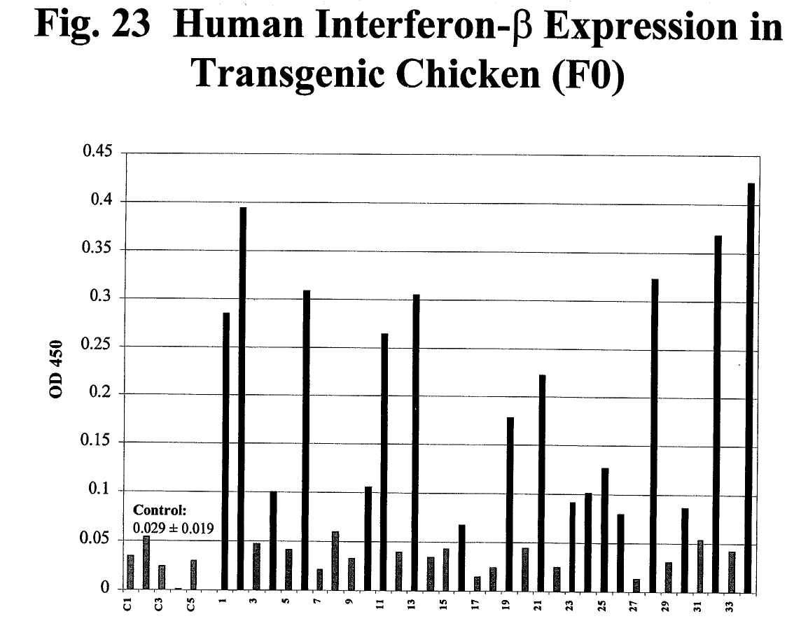

Based on PCR analysis, 11% (10/90) of all samples analyzed were found to contain a 230 bp fragment of pGL-3 control DNA (FIG. 22a). Furthermore, the PCR products in the agarose gel were blotted to a membrane for southern blot analysis using the ³²P labeled oligo probe. Hybridization of the oligo probe to the PCR products resulted in 49% (44/90) of the samples having a hybridization signal (FIG. 22b), indicating that some of the transgene or exogenous DNA may be present at low copy number. No hybridization signal was detected in these ninety chicken using genomic DNA samples by Southern blot analysis. The lack of hybridization signal in genomic Southern blot analysis may be due to a high degree of mosaicism in these transgenic chicken embryos, and /or low copy number of integration by the pGL-3 plasmid.

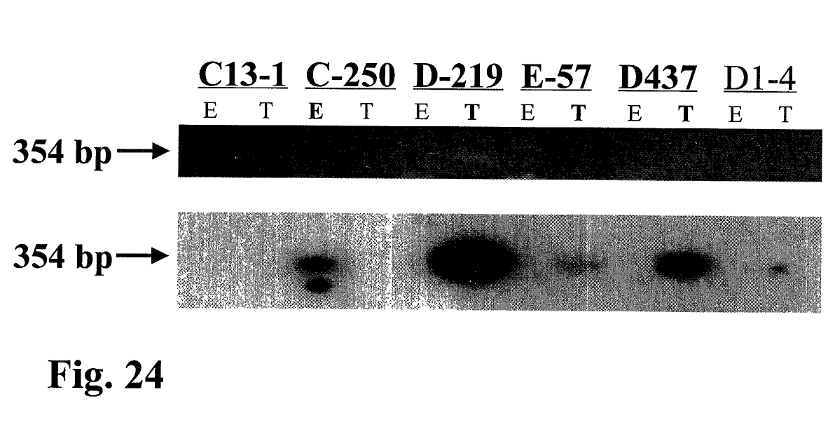

Furthermore, a gene for the human interferon-β was also introduced into F₀ chickens and was shown to be expressed in the F₀ chickens. Briefly, human intereferon-β in a plasmid vector and driven by a chicken cytoplasmic β-actin promoter was incubated with chicken sperm cells linked with mAb C, as prepared and described above, and artificially inseminated into hens. After the F₀ generation chicks were hatched and grown to about 3-5 weeks old, sera from the F₀ generation chickens were extracted and analyzed for human interferon-β using ELISA according to the protocols and kit manufactured by PBL Biomedical Laboratories, New Brunswick, N.J. Duplicate chicken sera were analyzed and the average levels of human interferon-β are shown in FIG. 24.

FIG. 24 shows the expression of human interferon-D in F₀ generation chickens, wherein the y-axis shows the optical density reading at 450 nm, and the x-axis provides for the number assigned to the chicken. As can be seen from the figure, control level (C1-C5) of human interferon β from non-transgenic chickens averaged around 0.029±0.019 OD₄₅₀ units. The black bars (chicken 1, 2, 4, 6, 10, 11, 13, 16, 19, 21, 23, 24, 255, 26, 28, 30, 32, and 34) show significant expression of human interferon-β above the control. This amounts to 53% (18/34) of the F₀ generation chickens expressing human interferon-β at a level significantly higher than the control group.

Interferon expressed in chickens may be useful in increasing the resistance of the chicken to viral infections. It may also be useful as a means of producing human interferon by expressing interferon in the eggs and purifying interferon from the eggs using methods known in the art. Other genes and proteins may similarly be expressed in the eggs for production and purification.

### EXAMPLE X

This example illustrates the introduction of DNA into and/or genetic modification of sperm cells attached with plasmid DNA.

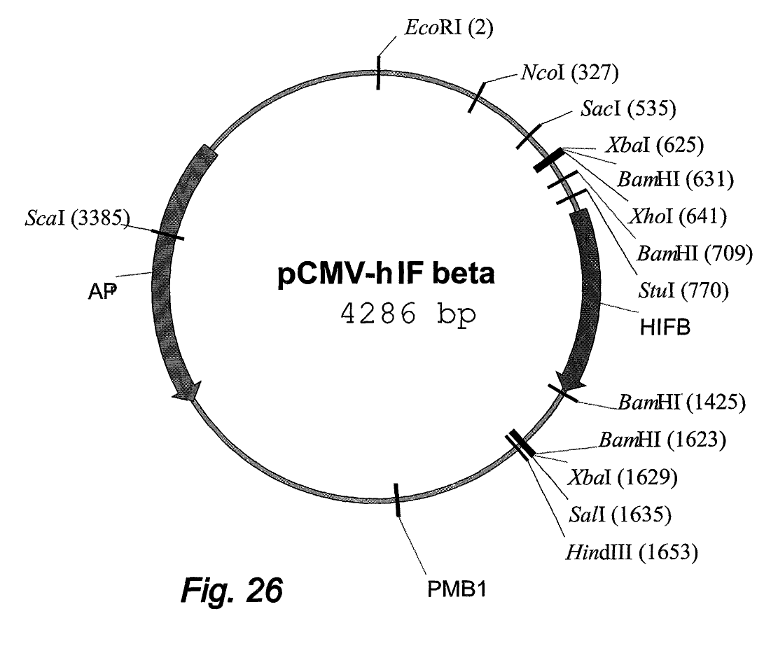

Ejaculated sperm cells from male bulls were prepared by diluting the cells with extender medium (Merck, Darmstadt Germany) to about 10⁶-10⁷ cells in a 1.5 ml microcentrifuge tube. Afterwards, three micrograms of mAbC antibody was added to the sperm cells and incubated at room temperature for about 40 minutes, with gentle mixing about every 10 minutes. The sperm cells were then spun at 2,000 rpm for three minutes to remove the supernatant and washed with 0.3 ml of extender medium having 1.5 mg/ml BSA. The sperm cells were again spun at 2,000 rpm for 3 minutes and resuspended in 0.4 ml extender medium with 1.5 mg/ml BSA. 1.5 micrograms of the pCMV-hlFbeta plasmid (FIG. 26), which comprises a human interferon β]gene driven by a CMV promoter similar to the pCMV β-gal plasmid, was then added to the sperm cells at room temperature and incubated for 40 minutes with gentle mixing every 10 minutes. The sperm cells were visually check under the microscope for sperm activity and number, before they were ariticially inseminated by inserting and injecting the sperm-linker-DNA mixture through the vagina of the cows.

After the F₀ generation cows were born and allowed to grow to 60 day old, genomic DNA was obtained from the ear and/or tail of the F₀ generation cows. Genomic DNA was analyzed using PCR for the presence of a 354 bp fragment from the plasmid using the following PCR primers: 5′-GTC AAT GGG AGT TTG TTT TG-3′ (forward bp 412-431) and 5′-GAA GTA AAG GCA ACA TCC AC-3′(reverse bp 746-765), which prime in the CMV enhancer and SV40 intron region of the pCMVP, respectively (Clontech Laboratories, Inc.). The amplification was carried as follows: 4 min at 95° C. followed by 40 cycles at 53° C. for 1 min, 72° C. for 1 min and 94° C. for 1 min, followed by 1 cycle at 53° C. for 1 min and 72° C. for 10 min. PCR samples were analyzed on a 1.5% agarose gel and transferred to a nylon membrane. A ³²P-end labeled third internal oligo (5′-AGC TCG TTT AGT GAA CCG TC-3′, bp 531-550) was used to verify the PCR product by hybridization.

FIG. 24 shows the results of the ethidium bromide staining and the results from hybridization of a ³²P end-labeled internal oligo. Possibly due to the mosaic nature of the introduction of the gene into the animal, the presence of the 384 bp fragment can be seen in the ear (E) tissue of C-250 and not in the tail tissue; while in D-219, E-57, and D437, the presence of the 384 bp fragment was detected in tail tissues and not in the ear tissues. Nevertheless, it is clear that artificial insemination of the sperm-linker-DNA complex results in animals which carries the plasmid DNA in their cells. D1-4 was included as a negative control with the ear and tail tissues coming from non-transgenic cows.

It should be noted that although this example uses a constitutively expressing promoter driving the expression of the interferon gene, inducible promoters or tissue specific promoters such as promoters for αS1-casein for production of interferon in milk may also be used.

### EXAMPLE XI

This example illustrates the introduction of DNA into and/or genetic modification of goats.

Female goats were prepared for artificial insemination as follows: On day one and between 8-10 am, female goats were implanted with EAZI-BREED CIDR (0.3 g Progesterone/each) from Pharmacia/UpJohn Pty Limited, Rydalmere NSW 2116. On day ten and at about 6:00 pm, the female goats were each injected with 5 ml of 500 I.U of Serum Gonadotropin (China Chemistry Drug Corp., Taipei, Taiwan) and 1 ml/each of Estrumate (263 micrograms of closprostenol sodium from Bayer Corp., Shawnee Mission, Kans.). On day 12, the CIDR were removed. On day 13 the estrus of the female goats were determined and the time recorded by observation of the animal behavior. Artificial insemination were then performed 23-25 hours later.

Ejaculated sperm cells from male goats were performed using the same procedure used with sperm cells from the cows. Similarly, the pCMV-hIFbeta plasmid (FIG. 26) was attached to the goat sperm cells and introduced into the animals by artificial insemination. PCR screening of the resulting F₀ generation goats were performed using the same protocol as in the F₀ cows.

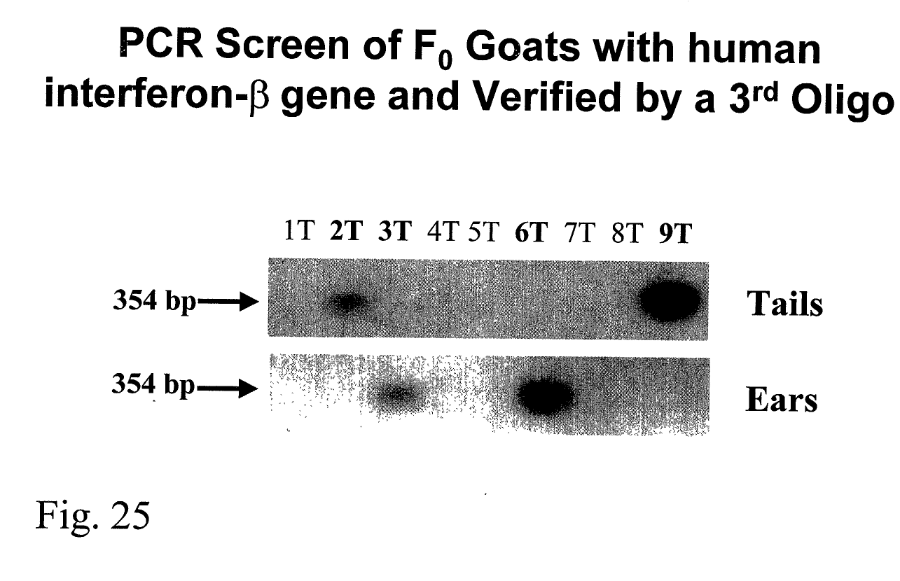

FIG. 25 shows the results of the PCR screening of the F₀ goats and hybridization with the ³²P-labeled internal oligo, similarly used with the F₀ cows. Similarly to the cows, the pCMV-hIFbeta plasmid (FIG. 26) has been introduced into the F₀ goats in a mosaic nature. For example, tails from F₀ goat numbers 2T and 9T show positive results of hybridization to a 354 bp PCR fragment while the ears from the same animals do not. On the other hand, ears from F₀ goat numbers 3T and 6T show positive results of hybridization while the tails from the same animals do not. Nevertheless, it is clear that artificial insemination of the sperm-linker-DNA complex results in animals which carries the plasmid DNA in their cells.

It should be noted that although this example uses a constitutively expressing promoter driving the expression of the interferon gene, inducible promoters or tissue specific promoters such as promoters for plactoglobulin for production of interferon in milk may also be used.

### EXAMPLE XII

The disclosed non-liposome based linker such as the mAbC antibody may also be used and provided in a kit for introduction of an exogenous DNA or RNA into an animal. The non-liposome based linker may be provided in a container such as a microcentrifuge tube, plates, or any other suitable containers for transport, storage, or binding to sperm cells. The kit, for example, may also contain instructions for performing the disclosed method of associating a polynucleotide such as a DNA with a sperm cell using the non-liposome based linker, and effecting in vitro or in vivo fertilization of a compatible animal's egg cell with the sperm-linker-DNA complex. The kit may also contain the sperm cells, the egg cells, and/or a control DNA molecule such as pSEAP-2 plasmid, pCMV-β plasmid, or pGL-3 plasmid.

### EXAMPLE XIII

The disclosed non-liposome based linker such as the mAbC antibody and method may also be used for functional screening of genes by the introduction of the gene into the animal and observing or analyzing the phenotype of the animal. For example, high throughput screening of gene function may be achieved by introducing genes of known or unknown functions into the animal by cloning the genes of interest into a suitable expression vector having constitutive expression such as pCR3.1 (Invitrogen, San Diego, Calif.) or tissue specific expression vectors such as vectors having smooth muscle alpha-actin promoter for vascular expression, cardiac myosin heavy chain promoters for expression in heart, for example, and other suitable expression promoters depending on the study.

Sperm cells from mice or any other animal may be provided in 24, 48, 96 well or any other suitable plate or container, and incubated with the non-liposome based linker according to the procedures described above in the various examples. Each gene of interest cloned in the suitable expression vector may then be incubated and linked with the sperm cells/non-liposome based linker complex. Afterwards, the sperm-linker-nucleic acid vector complex may then be transferred to another 24, 48, 96 well or any other suitable plate or container containing at least one, preferably multiple, egg cells, for in vitro fertilization. Once fertilization is complete, the resulting zygotes may then be transplanted into psuedo-pregnant animal for development. Gene function may then be elucidated based on the effect of the transgenes effect on the animal.

Alternatively, the sperm-linker-nucleic acid vector complex may also be used to achieve artificial insemination such as by directly injecting the complex into the vagina or oviduct of the female animal such as mice, rats, chickens, pigs, or cows. This removes the need for additional step of in-vitro fertilization and simplifies the procedures for high throughput functional screening.

Functional screening of genes may also involve the introduction of larger pieces of DNA such as sheared chromosomal DNA fragments comprising multiple genes (e.g. 10 genes) including their respective promoters and enhancers. The chromosomal DNA fragments may then be introduced into the animal by linking the DNA fragments to the sperm cells and used for in vitro or in vivo fertilization. Based on the resulting phenotype observed to be of interests, the gene responsible for the phenotype may be isolated from the animal, and further tested.

The preceding examples demonstrate that the inventor has produced a number of genetically modified animals using the sperm vector as described above. These data are intended only as examples and are not intended to limit the invention to these examples. It is understood that modifying the examples below does not depart from the spirit of the invention.

What is claimed is:

1. A method comprising the steps:
associating at least one polynucleotide molecule to a non-human sperm cell through at least one non-liposome based linker;
effecting in vitro or in vivo fertilization of a non-human egg cell with the non-human sperm cell associated through the non-liposome based linker with the at least one polynucleotide molecule.

2. The method of claim 1 wherein the associating occurred in vitro or in vivo.

3. The method of claim 1 wherein the non-liposome based linker further comprises a polypeptide.

4. The method of claim 3 wherein the polypeptide is a protein.

5. The method of claim 3 wherein the non-liposome based linker is associated to an external surface of the non-human sperm cell.

6. The method of claim 1 wherein the non-liposome based linker is a sperm-specific linker.

7. The method of claim 1 wherein the non-liposome based linker is an immunoglobulin.

8. The method of claim 1 wherein the non-liposome based linker is an antibody.

9. The method of claim 1 wherein the at least one polynucleotide molecule is a DNA molecule.

10. The method of claim 9 wherein the DNA molecule comprises a nucleic acid sequence encoding for a growth hormone

11. The method of claim 9 wherein the DNA molecule comprises a nucleic acid sequence encoding for a gene product that confers pathogen or disease resistance.

12. The method of claim 11 wherein the gene product is human interferon β.

13. The method of claim 9 wherein the DNA molecule comprises a nucleic acid sequence encoding for a gene product that confers immune tolerance in a human body.

14. The method of claim 9 wherein the DNA molecule comprises a nucleic acid sequence encoding for a gene product that is specifically produced in a non-human animal's milk.

15. The method of claim 1 wherein the non-liposome based linker interacts with the at least one polynucleotide molecule via molecular interaction from the group consisting of ionic interaction, covalent interaction, Vander Waals interaction, and ligand-receptor interaction

16. The method of claim 8 wherein the antibody is characterized by having binding affinity to a sperm cell and wherein the sperm cell bound with the antibody retains the ability to fertilize an oocyte.

17. The method of claim 8 wherein the antibody is characterized by ability to bind to sperm cells from a plurality of species of animals.

18. The method of claim 8 wherein the antibody exhibits binding properties to a polynucleotide such that upon fertilization, the polynucleotide is introduced into a zygote.

19. The method of claim 1 wherein the non-human sperm cell is selected from the group consisting of: mouse sperm cell, cow sperm cell, pig sperm cell, chicken sperm cell, sheep sperm cell, and goat sperm cell.

20. An article of manufacture comprising:
a container comprising a non-liposome based linker; and
an instruction to use the non-liposome based linker for attaching a polynucleotide molecule to a sperm cell.

21. The article of manufacture of claim 20 wherein the non-liposome based linker is an antibody.

22. The article of manufacture of claim 21 wherein the antibody is characterized by having binding affinity to a sperm cell and wherein the sperm cell bound with the antibody retains the ability to fertilize an oocyte.

23. The article of manufacture of claim 21 wherein the antibody is characterized by the ability to bind to sperm cells from a plurality of species of animals.

24. The article of manufacture of claim 21 wherein the antibody exhibits binding properties to a polynucleotide such that upon fertilization, the polynucleotide is introduced into a zygote.

25. The article of manufacture of claim 20 further comprising a second container that comprises the sperm cell.

26. The article of manufacture of claim 20 wherein the sperm cell is selected from the group consisting of mouse sperm cell, cow sperm cell, pig sperm cell, chicken sperm cell, sheep sperm cell, and goat sperm cell.

27. The article of manufacture of claim 20 further comprising a second container that comprises the polynucleotide molecule.

28. The article of manufacture of claim 20 wherein the instruction provide directions to perform the following steps:
associating at least one polynucleotide molecule to a non-human sperm cell through at least one non-liposome based linker;
effecting in vitro or in vivo fertilization of a non-human egg cell with the non-human sperm cell associated through the non-liposome based linker with the at least one polynucleotide molecule.

29. The article of manufacture of claim 20 further comprising a sperm cell, wherein the non-liposome based linker and the sperm cell are contained in the same container.

30. The article of manufacture of claim 29 wherein the container is adapted to receive a solution comprising a polynucleotide molecule.

31. The article of manufacture of claim 20 wherein the article of manufacture is a kit.

32. An article of manufacture comprising:
a container comprising an antibody, wherein the antibody is characterized by having binding affinity to a sperm cell and wherein the sperm cell bound with the antibody retains the ability to fertilize an oocyte.

33. The article of manufacture of claim 32 wherein the antibody is characterized by the ability to bind to sperm cells from a plurality of species of animals.

34. The article of manufacture of claim 32 wherein the antibody exhibits binding properties to a polynucleotide such that upon fertilization, the polynucleotide is introduced into a zygote.

35. The article of manufacture of claim 32 wherein the container further comprises a sperm cell, and wherein the antibody is bound to the sperm cell.

36. The article of manufacture of claim 32 wherein the container is adapted to receive a solution comprising a polynucleotide molecule.

37. The article of manufacture of claim 32 wherein the container is a plate having a plurality of wells.

38. The article of manufacture of claim 32 wherein the article of manufacture is a kit and further comprises a second container comprising non-human egg cell.

39. A chicken comprising a nucleic acid molecule, wherein the nucleic acid molecule comprises a nucleic acid sequence encoding human interferon.

40. The chicken of claim 39 wherein the human interferon is human interferon β.

41. The chicken of claim 39 wherein the nucleic acid molecule further comprises a β-actin promoter.

42. The chicken of claim 39 wherein the human interferon is present in serum of the chicken.

43. The chicken of claim 42 wherein the human interferon in the serum of the chicken is detectable by ELISA.

44. The chicken of claim 39 further comprising human interferon β proteins detectable by ELISA in a serum sample of the chicken.
